(19) United States
(12) Patent Application Publication
(10) Pub. No.: US 2004/0192600 A1
(43) Pub. Date: Sep. 30, 2004

(54) Combination of selective cox-2 inhibitor and lung surfactant for respiratory syndrome

(76) Inventor: Dietrich Hafner, Konstanz (DE)

Correspondence Address: NATH & ASSOCIATES PLLC, 1030 FIFTEENTH STREET, N.W., SIXTH FLOOR, WASHINGTON, DC, 20005, US

(21) Appl. No.: 10/485,157

(22) PCT Filed: Jul. 23, 2002
(86) PCT No.: PCT/EP02/08176

(30) Foreign Application Priority Data
Aug. 1, 2001 (EP) 01118540.2

(51) Int. Cl. A61K 38/17; A61K 31/685; A61K 31/415

(52) U.S. Cl. 514/12; 514/406; 514/78

(57) ABSTRACT

Novel compositions for the treatment of IRDS and ARDS are indicated which contain a selective COX-2 inhibitor, and/or a pharmaceutically acceptable salt thereof and lung surfactant.

### TECHNICAL FIELD

The invention relates to a novel composition for the treatment of disease conditions which are designated as Infant Respiratory Distress Syndrome (IRDS) and Acute or Adult Respiratory Distress Syndrome (ARDS).

### PRIOR ART

Adult Respiratory Distress Syndrome (ARDS) is a descriptive expression which is applied to a large number of acute, diffusely infiltrative pulmonary lesions of different etiology if they are associated with a severe gas exchange disorder (in particular arterial hypoxemia). The expression ARDS is used because of the numerous common clinical and pathological features with the Infant Respiratory Distress Syndrome (IRDS). If, in the case of IRDS, the lung surfactant deficiency caused by premature birth is predominant, then in the case of ARDS a lung surfactant malfunction is caused by a lung disorder based on differing etiologies.

Triggering causes for ARDS can, for example, be (cited in accordance with Harrison's Principles of Internal Medicine 14th Ed. 1998 McGraw-Hill Int. Book Comp.) diffuse pulmonary infections (for example due to viruses, bacteria, fungi), aspiration of, for example, gastric juice or in the case of near-drowning, inhalation of toxins or irritants (for example chlorine gas, nitrogen oxides, smoke), direct or indirect trauma (for example multiple fractures or pulmonary contusion), systemic reactions to inflammations outside the lung (for example hemorrhagic pancreatitis, gram-negative septicemia), transfusions of high blood volumes or alternatively after cardiopulmonary bypass.

With a mortality of 50-60% (survey in Schuster Chest 1995, 107:1721-1726), the prognoses of an ARDS patient are still to be designated as very unfavorable.

The therapy of ARDS consists mainly in the earliest possible application of different forms of ventilation [for example PEEP (positive end-expiratory pressure), using low tidal volumes (The ARDS Network Study Group: N. Engl. J. Med. 2000 342(18):1301-1308) raising of the oxygen concentration of the respiratory air, SIMV (Synchronized Intermittent Mandatory Ventilation; Harrison's Principles of Internal Medicine 14th Ed. 1998 McGraw-Hill Int. Book Comp.)] up to extracorporeal membrane oxygenation (ECMO; Zapol and Lemaire Adult Respiratory Distress Syndrome, Marcel Dekker Inc, 1991).

The targetted use of various ventilation techniques has only led to a certain lowering of mortality and includes the risk of setting in motion a vicious circle. By ventilation with pressure and high FiO₂ (Fraction of Inspired Oxygen; proportion of oxygen in the respiratory air), the lungs themselves can be damaged and as a result of this even higher pressures and higher FiO₂ may be required in order to obtain an adequate oxygenation of the blood.

Different approaches to the solution of the abovementioned problems are followed. These include lung surfactant substitution [survey, for example, B. Lachmann, D. Gommers and E. P. Eijking, Exogenous surfactant therapy in adults, Atemw.-Lungenkrkh. 1993, 19:581-591; T. J. Gregory et al., Bovine Surfactant Therapy for Patients with Acute Respiratory Distress Syndrome, Am. J. Respir. Crit. Care Med. 1997, Vol. 155, 1309-1315] up to purely antiinflammatory therapy with, for example, prostaglandin E₁ (PGE₁; Abraham et al., Crit Care Med 1996, 24:10-15) or glucocorticosteroids (Bernard et al., N. Engl. J. Med. 1987, 317:1565-1570). Although certain successes were achieved by the administration of lung surfactant (for example Walmrath et al., Am. J. Resp. Crit. Care Med. 1996, 154:57-62), the purely antiinflammatory therapies hitherto led to little if any success. There is a certain contrast here to the pathological or histopathological findings in ARDS. Thus, massive polymorphonuclear leucocyte infiltrations (survey, for example, Thiel et al., Anaesthesist 1996, 45:113-130) were found in the lungs and the lavage of patients with ARDS, and a number of inflammatory mediators are detectable. Still under investigation were PGE, in a liposomal intravenous administration form (Abraham et al., Crit. Care Med. 1996, 24:10-15) and substances which aim at the inhibition of phosphatidic acids (for example lisofylline; Rice et al., Proc. Natl. Acad. Sci. 1994, 91:3857-3861) or recombinant human interleukin 1 (IL-1) receptor antagonists (Fisher et al., JAMA 1994, 271:1836-1843). Both PGE₁ and the IL-1 receptor antagonist, however, are restricted in their therapeutic utility due to side effects, and the clinical studies were terminated or concluded without success. This was also true for a clinical trial that used ibuprofen (Bernard G. R. et al., N. Engl. J. Med. 1997; 336(13):912-8) as a non-selective cyclooxygenase (=COX)-inhibitor.

WO 9609831 indicates compositions for the treatment of ARDS and IRDS which contain a glucocorticosteroid and lung surfactant. WO 9835683 relates to a composition for the treatment of IRDS and ARDS comprising N-(3,5-dichloropyrid-4-yl)-3-cyclopropylmethoxy-4-difluoromethoxybenzamide and lung surfactant. WO 9966926 relates to a composition for the treatment of IRDS and ARDS comprising 4-(2,6-dichloroanilino)-3-thiopheneacetic acid or 2-(2-hydroxyethoxy)ethyl 4-(2,6-dichloroanilino)-3-thiopheneacetate and lung surfactant. EP-B-0451215 describes compositions for the administration of a pharmaceutically active compound via the lungs. These compositions include liposomes which contain a pharmaceutically active compound and a lung surfactant protein. EP-B-0055041 describes preparations for inhalation or infusion for the treatment of disorders of the respiratory organs, which contain an active compound against disorders of the respiratory organs and natural lung surfactant. Preparations for the treatment of ARDS or IRDS are not disclosed.

### DESCRIPTION OF THE INVENTION

It has now surprisingly been found that by the administration of a combination of a selective COX-2 inhibitor and lung surfactant a synergistic effect can be achieved in the treatment of IRDS and ARDS. Thus the treatment of IRDS and ARDS may be shortened and the high mortality accompanying these syndromes can be reduced.

The invention therefore provides a composition for the treatment of IRDS or ARDS comprising a selective COX-2 inhibitor and lung surfactant.

Further embodiments of the invention follow from the patent claims.

Selective COX-2 inhibitors according to the invention are active agents that selectively inhibit the enzyme cyclooxygenase-2 (COX-2) in preference to cyclooxygenase-1 (COX-1). Selective COX-2 inhibitors are of importance in the therapy of inflammation or inflammation related disorders. The ability of an active agent to treat cyclooxygenase mediated diseases can be demonstrated in an in vitro assay by measuring the amount of prostaglandin E.sub.2 (PGE.sub.2) synthesized in the presence of arachidonic acid, cyclooxygenase-1 or cyclooxygenase-2 and the active agent. Suitable assays for determination of cyclooxygenase activity of an active agent which may be mentioned are whole cell and microsomal cyclooxygenase assays. Such assays are disclosed for example in WO 9500501. These assays measure prostaglandin E.sub.2 (PGE.sub.2) synthesis in response to arachidonic acid, using a radio-immunoassay. Cells which may be used for whole cell assays, and from which microsomes may be prepared for microsomal assays, are human osteosarcoma 143 cells (which specifically express cyclooxygenase-2) and human U-937 cells (which specifically express cyclooxygenase-1). In these assays, 100% activity is defined as the difference between prostaglandin E.sub.2 synthesis in the absence and presence of arachidonate addition. IC.sub.50 values represent the concentration of inhibitor required to return PGE.sub.2 synthesis to 50% of that obtained as compared to uninhibited control. In connection with the present invention an active agent preferentially is said to selectively inhibit COX-2 in preference to COX-1 if the ratio of the IC.sub.50 concentration for COX-1 inhibition to COX-2 inhibition is 100 or greater. A further assay to determine COX-1 and COX-2 inhibiton is disclosed in WO 9515316.

The preparation of selective COX-2 inhibitors and their use in the treatment of inflammation or inflammation related disorders is described for example in WO 9515316, WO 9500501, WO 9638442, WO 9606840, WO 9636617, WO 9636623, WO 9803484, WO 9738986 and WO 9625405. Mention may preferably be made here of p-[5-p-tolyl-3-(trifluoromethyl)pyrazol-1-yl]benzenesulfonamide(INN: Celecoxib), 4-[p-(methylsulfonyl)phenyl]-3-phenyl-2(5H)-furanone(INN: Rofecoxib), p-[3-(difluoromethyl)-5-(3-fluoro-4-methoxyphenyl)pyrazol-1-yl]-benzenesulfonamide)INN: Deracoxib), 5-chloro-6′-methyl-3-[4-(methylsulfonyl)phenyl]-2,3′-bipyridine(INN: Etoricoxib), N-[[p-(5-methyl-3-phenyl-4-isoxazolyl)-phenyl]sulfonyl]propionamide(INN:Parecoxib), 4-(4-cyclohexyl-2-methyloxazol-5-yl)-2-fluorobenzenesulfonamide(INN: Tilmacoxib) and p-(5-methyl-3-phenyl-4-isoxazolyl)benzenesulfonamide(Valdecoxib). The selective COX-2 inhibitors are present here as such or in the form of a pharmaceutically acceptable salt thereof.

Natural lung surfactant has surface-active properties; for example, it reduces the surface tension in the pulmonary alveolae. A simple and fast in vitro test for the determination of the surface activity of lung surfactant is, for example, the so-called Wilhelmy balance [Goerke, J. Biochim. Biophys. Acta, 344: 241-261 (1974), King R. J. and Clements J. A., Am. J. Physicol. 223: 715-726 (1972)]. This method gives indications about the quality of the lung surfactant, measured as the action of a lung surfactant to achieve a surface tension of almost zero mN/m.

In the test with the Wilhelmy balance, a suspension of lung surfactant having a defined phospholipid concentration is injected into an aqueous solution. The phospholipids are distributed at the gas/liquid phase boundary, forming a so-called monolayer. This monolayer reduces the surface tension of the water. A small platinum sheet is carefully immersed in the solution. The force which pulls the platinum sheet down can now be determined using sensitive transformers. This force is proportional to the surface tension and depends on the size of the platinum sheet. Another measuring device for determining the surface activity of lung surfactant is the “Pulsating Bubble Surfactometer” (Possmayer F., Yu S. and Weber M., Prog. Resp. Res., Ed. v. Wichert, Vol. 18: 112-120 (1984)].

The activity of a lung surfactant composition can also be determined by in vivo tests. Indications about the activity of a lung surfactant can be obtained by measuring, for example, lung compliance, blood gas exchange or the required respiratory pressures.

Lung surfactant is understood according to the invention as meaning the numerous known compositions and their modifications which have the function of natural lung surfactant. In this case, preferred compositions are those which, for example, have activity in the tests described above. Particularly preferred compositions are those which exhibit increased activity in such a test in comparison with natural, in particular human, lung surfactant. In this context, these can be compositions which only contain phospholipids, but also compositions which, apart from the phospholipids, inter alia additionally contain lung surfactant protein. Preferred phospholipids according to the invention are dipalmitoylphosphatidylcholine (DPPC), palmitoyloleylphosphatidylglycerol (POPG) and/or phosphatidylglycerol (PG). Particularly preferably, the phospholiplds are mixtures of various phospholipids, in particular mixtures of dipalmitoylphosphatidylcholine (DPPC) and palmitoyloleylphosphatidylglycerol (POPG), preferably in the ratio from 7 to 3 to 3 to 7. Commercial products which may be mentioned are Curosurf® (Serono, Pharma GmbH, Unterschleiβheim), a natural surfactant from homogenized porcine lungs, Survanta® (Abbott GmbH, Wiesbaden) and Alveofact® (Boehringer Ingelheim), both extracts of bovine lungs, as well as Exosurf® (Glaxo Wellcome), a synthetic phospholipid containing excipients. Suitable lung surfactant proteins are both the proteins obtained from natural sources, such as pulmonary lavage or extraction from amniotic fluid, and the proteins prepared by genetic engineering or chemical synthesis. According to the invention, in particular the lung surfactant proteins designated by SP-B and SP-C and their modified derivatives are of interest. The amino acid sequences of these lung surfactant proteins, their isolation or preparation by genetic engineering are known (e.g. from WO 8603408, EP-A-0251449, WO 8904326, WO 8706943, WO 8803170, WO 9100871, EP-A-0368823 and EP-A-0348967). Modified derivatives of the lung surfactant proteins designated by SP-C, which differ from human SP-C by the replacement of a few amino acids, are described, for example, in WO 9118015 and WO 9532992. Particularly to be emphasized in this connection are the recombinant SP-C derivatives which are disclosed in WO 9532992, in particular those which differ from human SP-C in positions 4 and 5 by the replacement of cysteine by phenylalanine and in position 32 by the replacement of methionine by isoleucine [designated below as rSP-C (FF/I) or Iusupultide (INN)]. Modified derivatives of lung surfactant proteins are also understood as meaning those proteins which have a completely originally designed amino acid sequence with respect to their lung surfactant properties, such as are described in EP-A-0593094 and WO 9222315. Preferably, the polypeptide KL4 (INN: sinapultide) may be mentioned in this connection. According to the invention lung surfactant can also comprise mixtures of different lung surfactant proteins. In EP-B-0100910, EP-A-0110498, EP-B-0119056, EP-B-0145005 and EP-B-0286011, phospholipid compositions with and without lung surfactant proteins are described which are likewise suitable as components of the preparations. As further constituents which can be present in lung surfactant preparations, fatty acids such as palmitic acid may be mentioned. The lung surfactant preparations can also contain electrolytes such as calcium, magnesium and/or sodium salts (for example calcium chloride, sodium chloride and/or sodium hydrogencarbonate) in order to establish an advantageous viscosity.

Lung surfactant preparations can be prepared by processes known per se and familiar to the person skilled in the art, for example as described in WO 9532992. According to the invention, the lung surfactant preparations are preferably lyophilized and in particular spray-dried lung surfactant preparations. Lyophilized preparations are disclosed, for example, in WO 9735882, WO 9100871 and DE 3229179. WO 9726863 describes a process for the preparation of powdered lung surfactant preparations by spray drying. According to the invention, lung surfactant preparations prepared in this way are preferred. Preferred lung surfactant preparations according to the invention contain 80 to 95% by weight of phospholipids, 0.5 to 3.0% by weight of lung surfactant proteins, 3 to 15% by weight of fatty acid, preferably palmitic acid, and 0 to 3% by weight of calicum chloride.

Moreover, the present invention provides pharmaceutical compositions for the treatment and/or prophylaxis of IRDS or ARDS, which comprise a selective COX-2 inhibitor or a pharmaceutically acceptable salt, thereof, and lung surfactant. The pharmaceutical compositions of the present invention may also contain further pharmaceutical auxiliaries (such as a pharmaceutically acceptable carrier) and optionally other therapeutic ingredients. Preferably the compositions according to the invention are made available either in liquid form for intratracheal or intrabronchial administration or in powder form for administration by inhalation. The compositions are prepared by procedures familiar to those skilled in the art, if appropriate using further suitable pharmaceutical auxiliaries. A powder form is obtained, for example, by mixing liquid lung surfactant preparations, for example aqueous suspensions, with aqueous solutions or suspensions of a selective COX-2 inhibitors and then lyophilizing and micronizing it. Alternatively, a solution of a lung surfactant and a selective COX-2 inhibitors can be lyophilized in a suitable solvent, such as, for example, tert-butanol, and then micronized. Spray-drying of a mixture of an aqueous lung surfactant suspension and an aqueous solution or suspension of a selective COX-2 inhibitor or a solution of a lung surfactant and a selective COX-2 inhibitor in suitable solvents, such as alcohols (for example methanol, ethanol, 2-propanol), chloroform, dichloromethane, acetone and their mixtures, which optionally can additionally contain small amounts of water, also leads to powdered preparations. Administration by inhalation can also be carried out by atomizing solutions or suspensions which contain the compositions according to the invention.

Below, the preparation of a powdered preparation by spray-drying is described by way of example:

### EXAMPLE 1

5.46 g of 1,2-dipalmitoyl-3-sn-phosphatidylcholine, 2.31 g of 1-palmitoyl-2-oleoyl-3-sn-phosphatidylglycerolammonium, 0.9 g of a selective COX-2 inhibitor, 0.38 g of palmitic acid, 0.21 g of calcium chloride and 0.15 g of rSP-C [recombinant lung surfactant protein SP-C (FF/I)] are dissolved in 500 ml of 2-propanol/water (90:10) and spray-dried in a laboratory spray drier Büchi B 191. Spray conditions: the gas for drying is nitrogen, the inlet temperature is 110° C., the outlet temperature is 58-62° C. This gives a fine white powder.

### EXAMPLE 2

5.46 g of 1,2-dipalmitoyl-3-sn-phosphatidylcholine, 2.31 g of 1-palmitoyl-2-oleoyl-3-sn-phosphatidylglycerolammonium, 0.09 g of a selective COX-2 inhibitor, 0.38 g of palmitic acid, 0.21 g of calcium chloride and 0.15 g of rSP-C [recombinant lung surfactant protein SP-C (FF/I)] are dissolved in 500 ml of 2-propanol/water (90:10) and spray-dried in a laboratory spray drier Büchi B 191. Spray conditions: the gas for drying is nitrogen, the inlet temperature is 110° C., the outlet temperature is 58-62° C. This gives a fine white powder.

The invention also provides a method for treating mammals, including humans, suffering from IRDS or ARDS. The method comprises administering a therapeutically effective and pharmacologically tolerable amount of one of the compositions according to the invention to the diseased mammal. The invention furthermore provides the compositions according to the invention for use in the treatment of IRDS and ARDS.

The amount of selective COX-2 inhibitor and lung surfactant that is administered and the dosage regimen for treating a disease condition with the compositions of this invention depends on a variety of factors, including the age, weight, sex and medical condition of the subject, the severity of the disease, the route and frequency of administration, and the particular compound employed, and thus may vary. The pharmaceutical compositions according to the invention preferentially contains the selective COX-2 inhibitors in amounts common in the treatment of inflammatory diseases or diseases related to inflammation. Thus the compositon according to the invention may contain the selective COX-2 inhibitor in the range of about 0.1 to 2000 mg, preferably in the range of about 0.5 to 500 mg and most preferably between about 1 and 100 mg. A daily dose of about 0.01 to 100 mg/kg body weight, preferably between about 0.1 and about 50 mg/kg body weight and most preferably from about 1 to 20 mg/kg body weight, may be appropriate. Dosage levels of the order of from about 0.01 mg to about 140 mg/kg of body weight per day are useful in the treatment of the above-indicated conditions, or alternatively about 0.5 mg to about 7 g per patient per day. The composition according to the invention preferentially contains the lung surfactant in such amounts that the amount of phospholipids is between 12.5 and 200 mg per kilogram of body weight per application. Compositions according to the invention advantageously contain 0.5 to 30 percent by weight of a selective COX-2 inhibitors and 70 to 99.5 percent of weight of lung surfactant.

The composition according to the invention is administered in a manner known to the person skilled in the art, preferably by intratracheal instillation (infusion or bolus) of a solution or suspension of the composition or in the form of an atomization of a solution or suspension of the composition or by atomization of composition in powder form. Preferably, the compositions according to the invention for administration are dissolved or suspended in a suitable solvent or resuspension medium, in particular if the compositons are present in lyophilized or spray-dried form. Preferably, the suitable resuspension medium is a physiological saline solution. It has proven advantageous to administer suspensions or solutions of the compositions according to the invention which contain 12.5 to 100 mg of phospholipids per ml of suspension. Preferably, the compositions according to the invention are administered per application in such an amount that the amount of phospholipids is between 12.5 and 200 mg per kilogram of body weight. Preferentially, administration is carried out 1 to 4 times daily over a period of 1 to 7 days. A process is preferred in which the solution or suspension of the composition employed contains 0.5 to 2.0 mg of rSP-C (FF/I) per ml of solvent. Particular mention may be made of a process in which the solution or suspension employed contains 0.75 to 1.5 mg of rSP-C (FF/I) per ml of solvent or suspension medium.

The compositions according to the invention are administered, for example, 3 to 4 times daily for 2 to 4 days. For example, compositions comprising 6 mg of a selective COX-2 inhibitor, and 50 mg of phospholipids are administered 6 times at an interval of 6 hours by inhalation or intratracheally or intrabronchially.

A further subject of the invention is a commercial product comprising a customary secondary packaging, a primary packaging comprising a pharmaceutical composition (for example an ampoule) and, if desired, a pack insert, the pharmaceutical composition comprising a selective COX-2 inhibitor and/or a salt thereof and lung surfactant, the composition being suitable for the prophylaxis and/or treatment of ARDS or IRDS and reference being made on the secondary packaging or on the pack insert of the commercial product to the suitability of the pharmaceutical compositon preparation for the prophylaxis and/or treatment of ARDS or IRDS. The secondary packaging, the primary packaging comprising the pharmaceutical composition and the pack insert otherwise correspond to what the person skilled in the art would regard as standard for pharmaceutical compositions of this type. Suitable primary packagings are, for example, ampoules or bottles of suitable materials such as transparent polyethylene or glass or alternatively suitable means of administration such as are customarily employed for the administration of active compounds into the lungs. By way of example, mention may be made of means of administration for the atomization of an active compound solution or suspension or for the atomization of active compound powder. Preferably, the primary packaging is a glass bottle which can be sealed, for example, by a commercially available rubber stopper or a septum. A suitable secondary packaging which may be mentioned by way of example is a folding box.

1. A composition for the treatment of IRDS and ARDS comprising a selective COX-2 inhibitor and/or a pharmaceutically acceptable salt thereof and lung surfactant.

2. A composition as claimed in claim 1, wherein, as lung surfactant, mixtures of phospholipids are contained.

3. A composition as claimed in claim 2, wherein phospholipids occurring in natural lung surfactant are contained.

4. A composition as claimed in claim 2 or 3, wherein lung surfactant proteins are additionally contained.

5. A composition as claimed in claim 4, wherein SP-B and/or SP-C and/or their modified derivatives are contained.

6. A composition as claimed in claim 1, wherein lung surfactants obtained by pulmonary lavage are contained.

7. The use of a specific COX-2 inhibitor, and/or a pharmaceutically acceptable salt thereof and lung surfactant for the production of medicaments for the treatment and/or prophylaxis of IRDS and ARDS.

9. The use of a composition as claimed in claim 1 for the treatment of IRDS and ARDS.
